(19) United States
(12) Patent Application Publication
(10) Pub. No.: US 2011/0039809 A1
(43) Pub. Date: Feb. 17, 2011

(54) COMBINED USE OF 25-HYDROXY-VITAMIN D3 AND VITAMIN D3 FOR IMPROVING BONE MINERAL DENSITY AND FOR TREATING OSTEOPORISIS

(76) Inventors: Neil Robert Buck, Leymen (FR); Wouter Claerhout, Singapore (SG); Bruno H. Leuenberger, Rheinfelden (CH); Elisabeth Stoecklin, Arlesheim (CH); Kai Urban, Bad Sackingen (DE); Swen Wolfram, Waldshut-Tiengen (DE)

Correspondence Address: NIXON & VANDERHYE, PC, 901 NORTH GLEBE ROAD, 11TH FLOOR, ARLINGTON, VA, 22203, US

(21) Appl. No.: 12/867,316

(22) PCT Filed: Feb. 12, 2009
(86) PCT No.: PCT/EP2009/051639

§ 371 Date: Nov. 1, 2010

Related U.S. Application Data
(60) Provisional application No. 61/028,510, filed on Feb. 13, 2008.
(60) Provisional application No. 61/031,671, filed on Feb. 26, 2008.
(60) Provisional application No. 61/036,922, filed on Mar. 14, 2008.
(60) Provisional application No. 61/036,928, filed on Mar. 15, 2008.

(51) Int. Cl. A61K 31/593; A61P 19/08; A61P 19/10

(52) U.S. Cl. 514/167

(57) ABSTRACT

We disclose the combined use of vitamin D (cholecalciferol) and 25-OH D3 (calcifediol) to treat and/or prevent osteoporosis. One or more bisphosphonate compounds to inhibit bone resorption may also be used. Forms and dosages of a pharmaceutical composition, as well as processes for manufacturing medicaments, are also disclosed.

### FIELD OF THE INVENTION

The present invention relates to use of vitamin D3 (cholecalciferol) and 25-hydroxyvitamin D3 (25-OH D3); calcifediol) to treat and/or prevent osteoporosis, and to lessen the severity of loss of bone density.

### BACKGROUND

Vitamin D (e.g., ergocalciferol and cholecalciferol) is a group of fat-soluble compounds defined by their biological activity. A deficiency of vitamin D causes rickets in children and osteomalacia in adults. But toxicity can occur after chronic intake of more than 100 times the recommended daily allowance (i.e., 5-15 μg or 200-600 IU vitamin D) for several months. For vitamin D, “The threshold for toxicity is 500 to 600 mcg/kg body weight per day. In general, adults should not consume more than three times the RDA for extended period of time” (Garrison & Somer, The Nutrition Desk Reference, Third Ed., McGraw-Hill, pg. 82, 1997). Hypercalcemia may occur at a blood concentration of 25-hydroxyvitamin D greater than 375 nmol/L. More recently, a safe upper level of Vitamin D was identified to be at least 250 μg/day (10′000 IU) (Hathcock et al. Am. J. Clin. Nutr. 85:6-18, 2007). Ingestion of such as a dietary supplement has been shown to result in a blood concentration of about 200 nmol/L 25-hydroxyvitamin D.

Vitamin D is a prohormone which has to be hydroxylated in the liver to produce 25-hydroxyvitamin D (calcifediol; 25-OH vitamin D; 25-OH D), which then undergoes another hydroxylation in the kidney and other tissues to produce 1,25-dihydroxyvitamin D, the active hormone form of vitamin D. 1,25-dihydroxyvitamin D is released into the blood, binds to vitamin D binding protein (DBP), and is transported to target tissues. Binding between 1,25-dihydroxyvitamin D and vitamin D receptor allows the complex to act as a transcription factor in the cell's nucleus.

Vitamin D deficiency may promote resorption of bone. It may also modulate function of the cardiovascular, immune, and muscular systems. Epidemiological studies find associations between vitamin D intake and its effect on blood pressure or glucose metabolism. The activity of vitamin D is under negative feedback control by parathyroid hormone.

Both Vitamin D and 25-OH D3 have been administered as pharmaceuticals in the past. Vitamin D, is of course widely available; 25-OH D3 was previously sold in the USA by Organon USA under the name “CALDEROL”, but is currently on the FDA's list of discontinued drugs. It was a gelatine capsule containing corn oil and 25-OH D3.

A liquid form of 25-OH D3 is currently sold in Spain by FAES Farma under the name “HIDROFEROL” in an oil solution.

The combination of vitamin D and 25-OH D3 has been used in animal feed. 25-OH D3 for use in feed is commercially available from DSM under the name “ROVIMIX HY-D”.

Tritsch et al. (US 2003/0170324) disclose a feed premix composition of at least 25-OH D3 in an amount between 5% and 50% (wt/wt) dissolved in oil and an antioxidant, an agent encapsulating droplets of 25-OH D3 and oil, and a nutritional additive (e.g., Vitamin D3). The premix may be added to poultry, swine, canine, or feline food. This composition stabilizes 25-OH D3 against oxidation.

Simoes-Nunes et al. (US 2005/0064018) discloses adding a combination of 25-OH Vitamin D3 and Vitamin D3 to animal feed. In particular, about 10 μg/kg to about 100 μg/kg of 25-OH Vitamin D3 and about 200 IU/kg to about 4,000 IU/kg of Vitamin D3 are added to swine feed. This addition improves the pig's bone strength.

Stark et al. (U.S. Pat. No. 5,695,794) disclose adding a combination of 25-OH Vitamin D3 and Vitamin D3 to poultry feed to ameliorate the effects of tibial dyschondroplasia.

Borenstein et al U.S. Pat. No. 5,043,170 discloses the combination of Vitamin D3 and either 1-alpha-hydroxycholecalciferol or 1 alpha, 25-dihydroxycholecalciferol to improve egg strength and leg strength in laying hens and older hens.

Chung et al, WO 2007/059960 discloses that sows fed a diet containing both Vitamin D3 and 25-hydroxVitamin D3 had improved general health status, body frame, litter size and health, and other production parameters. Also a 25-OH D3 human food supplement is disclosed, but its dosage range, 5-15 micrograms per kg body weight, which equals to an extremely high daily dosage of 300-900 micrograms per human is very high.

Bisphosphonate compounds, which are metabolically stabilized analogs of pyrophosphate, have been approved as effective for the treatment of osteoporosis by preventing the breakdown of bone (i.e., anti-resorptive agents). Bisphosphonate compounds adsorb to hydroxyapatite, which is a crystalline form of calcium and phosphate, in bone. Thus, a bisphosphonate compound prevents bone resorption by inhibiting the mobilization of calcium.

Francis (U.S. Pat. No. 4,230,700 and U.S. Pat. No. 4,330,537) discloses inhibiting bone resorption by a combination of etidronate and from about 100 IU to about 50,000 IU of a vitamin D-like compound.

Fleshner-Barak (WO 03/007916) discloses administration of bisphosphonate compound and natural vitamin D derivative such as 1,25-dihydroxyvitamin D3 or 24,25-dihydroxyvitamin D3, or 25-OH vitamin D3.

Daifotis et al. (WO 03/086415) disclose inhibiting bone resorption by a combination of at least one bisphosphonate compound and from about 100 IU to about 60,000 IU of a nonactivated metabolite of vitamin D2 and/or vitamin D3.

The aforementioned documents did not teach or suggest that the use of the combination vitamin D3 and 25-OH D3 would be surprisingly beneficial to treat and/or prevent osteoporosis in a human. Forms and dosages of the composition provide desirable effects on bone metabolism. Other advantages and improvements are described below or would be apparent from the disclosure herein.

### DETAILED DESCRIPTION OF THE INVENTION

It has been found that a combination of vitamin D3 (cholecalciferol) and 25-OH D3 (calcifediol), for use as a medicament, nutraceutical or food for bone health in a human has advantages over the administration of either vitamin D3 alone or 25-hydroxyvitamin D3 alone. The human may be any age, including children and juveniles, starting from birth to adulthood, and from 18 years to 80 years of age, or more than 80 years of age.

In a first aspect, one or more pharmaceutical, nutraceutical or food compositions suitable for human use are provided to administer both vitamin D3 and 25-OH D3 and a pharmaceutically acceptable carrier and thereby maintain bone health and/or treat and/or prevent osteoporosis.

This invention is also drawn to a human pharmaceutical composition for treatment of bone diseases and/or the maintenance of bone health, wherein the active ingredients consist essentially of a combination of Vitamin D and 25-OH D3.

In another aspect, a kit is provided which is comprised of multiple, separate dosages of the composition of vitamin D3 and 25-OH D3. They may be enclosed in a container: e.g., bottle, blister pack, or vial rack. Optionally, one or more other osteoporosis medicaments such as bisphosphonate compounds may be enclosed in the container. Further, instructions for administering the composition as a dosage to a human are provided within the kit.

In another aspect, a method of administering at least vitamin D3 and 25-hydroxy vitamin D3 to a human is provided to treat and/or prevent osteoporosis. They may be administered once per day, once per week, or once per month.

Vitamin D3 and 25-OH D3 can be co-administered with or without other osteoporosis medicaments, in combination or via separate formulations and not necessarily at the same time. “Other osteoporosis medicaments” as used herein refers to other compounds which may be administered to relieve, ameliorate, prevent, delay onset or the like of osteoporosis. Examples include: bisphosphonates, monoclonal antibodies, calcium forms, estrogens, phytoestrogens, and the like.

Another aspect of this invention is a bone health promoting food, functional food, food supplement or nutraceutical suitable for human consumption containing 25-OH D3, and preferably a combination of Vitamin D and 25-OH D3. In another embodiment, the 25-OH D3, alone or in combination with Vitamin D is the bone health promoting active ingredient in a food, functional food, food supplement or nutraceutical suitable for human consumption. The dosages of the 25-OH and/or D3 may be the same as those present in the pharmaceutical product, but preferably will tend towards the lower ranges. The food supplements and nutraceuticals may be in the form of tablets, capsules or other convenient dosage forms. The food may be a beverage or food, and if desired, may also contain other nutritionally effective compounds such as other vitamins, minerals, and the like.

As used throughout the specification and claims, the following definitions apply:

“Bone health” is meant to be a broad term. Maintainance of bone health is meant to encompass prevention of osteoporosis/osteopenia, rickets/osteomalasia, maintaining normal bone resorption/formation and regeneration events, maintaining normal Ca+ metabolism and preventing abnormal mobilization, and increasing peak bone mass.

“Vitamin D” means either Vitamin D3 (cholecalciferol) and/or Vitamin D2 (ergocalciferol). Humans are unable to make Vitamin D2 (ergocalciferol), but are able to use it as a source of Vitamin D. Vitamin D2 can be synthesized by various plants and is often used in Vitamin D in supplements as an equivalent to Vitamin D.

“Vitamin D metabolite” means any metabolite of Vitamin D other than 25-hydroxy vitamin D3.

“25-OH D3” refers specifically to 25-hydroxyvitamin D3.

“25-OH D” refers to the 25-hydroxylated metabolite of either Vitamin D2 or Vitamin D3 which is the major circulating form found in plasma.

“Prevent” is meant to include amelioration of the disease, lessening of the severity of the symptoms, early intervention, and lengthening the duration of onset of the disease, and is not intended to be limited to a situation where the patient is no longer able to contract the disease nor experience any symptoms.

“Bisphosphonates” include alendronate, clodronate, etidronate, ibandronate, olpadronate, minodronate, pamidronate, risedronate, tiludronate, and zoledronate.

“Other osteoporosis medicaments” as used herein refers to other compounds which may be administered to relieve, ameliorate, prevent, delay onset or the like of osteoporosis. Examples include: bisphosphonates, monoclonal antibodies, calcium forms, estrogens, phytoestrogens and the like.

Vitamin D deficiency is a leading contributor to bone health problems. Vitamin D deficiency/insufficiency is an especially prevalent condition in the elderly population and those who suffer chronic immobility regardless of age. Furthermore infants, toddlers, children, adolescences and young adults can suffer from hidden vitamin D deficiency. This may be due to the general lack of exposure to sunlight, a lessened ability of the body to manufacture vitamin D or metabolize it efficiently, or a number of other causes such as use of sunblockers whenever outside. Thus one aspect of this invention is the use of the combination of Vitamin D and 25-OH D3 to promote bone health in an elderly population. As used throughout, the term “elderly” is meant to encompass those individuals who are over 65 years of age, preferably over 70, and even over 80.

In another embodiment, this combination of 25-OH D3 and Vitamin D is suitable for maintaining bone health in people who are at risk of developing conditions characterized by Vitamin D deficiency or insufficiency. This would include especially all adults, including post-menopausal women (i.e. about age 45 and older) and men who are about age 45 and older. It is especially suitable for maintaining bone health in individuals who do not receive a great deal of natural sunlight exposure, such as for people who traditionally wear long clothing, do not go out of doors regularly, or who use sunscreens when they are exposed to sunlight, or live in geographical areas significantly north or south of the equator, where sunlight is less intense.

In another embodiment, the combination of 25-OH D3 and Vitamin D is suitable for improving bone health in children and young adults who are in the modeling phase of building bones. This is particularly a concern if they are at risk of Vitamin D deficiency or insufficiency before reaching peak bone mass. It is especially suitable for improving bone health in individuals before peak bone mass who do not receive a great deal of natural sunlight exposure, such as for the population who traditionally wear long clothing, do not go outdoors regularly, who use sunscreens when they are exposed to sunlight, or live in geographical areas significantly north or south of the equator, where sunlight is less intense. Thus another aspect of this invention is the use of the combination of 25-OH D3 and Vitamin D3 to increase peak bone mass in individuals who are in the modeling phase of building bone.

Another aspect of this invention is a method of maintaining bone health in persons with a malabsorption syndrome (e.g., affected by celiac disease, sprue, or short bowel syndrome) by administering the combination of Vitamin D and 25-OH D3.

Another aspect of this invention is a method of maintaining bone health in persons with impaired liver function, wherein the person cannot efficiently process Vitamin D into 25-hydroxyvitamin D by providing the person with a combination of Vitamin D and 25-hydroxyvitamin D3.

Vitamin D3 and 25-OH D3 may be obtained from any source, and a composition thereof may be prepared using convenient technology. In general, crystals of vitamin D3, 25-OH D3, or both (separately or together) are dissolved in an oil with heating and agitation. Preferably, the oil is transferred into a vessel and heated. Thereafter, vitamin D3, 25-OH D3, or both are added to the vessel, while maintaining the temperature of the oil or increasing it over time. The composition is agitated to dissolve the crystals of vitamin D3, 25-OH D3, or both. Prior to addition to the oil, the crystals may be reduced in size by milling and/or sieving, to enhance dissolving. The composition may be agitated by stirring, vessel rotation, mixing, homogenization, recirculation, or ultrasonication. Preferably, the oil may be heated in the vessel to a temperature from about 80° C. to about 85° C., sized crystals are introduced into the vessel, and the contents are stirred to dissolve the crystals into the oil.

The “oil” may be any edible oil, lipid, or fat: e.g., babassu oil, coconut oil, cohune oil, murumyru tallow, palm kernel oil, or tucum oil. The oil may be natural, synthetic, semisynthetic, or any combination thereof. Natural oil may be derived from any source (e.g., animal, plant, fungal, marine); synthetic or semisynthetic oil may be produced by convenient technology. Preferably, the oil is a mixture of plant medium chain triglycerides, mainly caprylic and capric acids. The composition may optionally contain one or more other suitable ingredients such as, for example, antioxidants, preservatives, dissolution agents, surfactants, pH adjusting agents or buffers, humectants, and any combination thereof. The foregoing are examples of pharmaceutically acceptable carriers.

Suitable antioxidants include tocopherol, mixed tocopherols, tocopherols from natural or synthetic sources, butylated hydroxy toluene (BHT), butylated hydroxy anisole (BHA), natural antioxidants like rosemary extract, propyl galate, and any others used in the manufacture of pharmaceuticals for humans. Preferably, the antioxidant is tocopherol. Suitable preservatives include methyl paraben, propyl paraben, potassium sorbate, sodium benzoate, benzoic acid, and any combination thereof. Suitable dissolution agents include inorganic or organic solvents: e.g., alcohols, chlorinated hydrocarbons, and any combination thereof. Suitable surfactants may be anionic, cationic, or nonionic: e.g., ascorbyl palmitate, polysorbates, polyethylene glycols, and any combination thereof. Suitable pH adjusting agents or buffers include citric acid-sodium citrate, phosphoric acid-sodium phosphate, acetic acid-sodium acetate, and any combination thereof. Suitable humectants include glycerol, sorbitol, polyethylene glycol, propylene glycol, and any combination thereof.

Once formed, the oil composition may be incorporated in various other useful compositions, some of which are discussed below. For example, emulsions may be formed, which may be optionally encapsulated or spray dried. A variety of emulsions may be prepared by combining the nonaqueous compositions described above with an aqueous composition. The emulsion may be of any type. Suitable emulsions include oil-in-water emulsions, water-in-oil emulsions, anhydrous emulsions, solid emulsions, and microemulsions. The emulsions may be prepared by any convenient technology. The emulsion contains an aqueous composition and a nonaqueous (e.g., oil) composition, wherein the latter comprises vitamin D3, 25-OH D3, or both (separately or together) dissolved in an oil in an amount of between about 3% and about 50% by weight based on the total weight of the oil composition. As used herein, “aqueous composition” and “aqueous phase” are used interchangeably. Generally, the emulsion may contain from about 20% to about 95% of an aqueous composition, and from about 5% to about 80% of a nonaqueous composition. Preferably, however, the emulsion contains from about 85% to about 95% (vol/vol) of an aqueous composition, and from about 5% to about 15% (vol/vol) of a nonaqueous composition. Conveniently, the nonaqueous composition may be dispersed as droplets in the aqueous composition. For example, the droplets may have a mean diameter of less than about 500 nm in the aqueous composition. Conveniently, the droplets have a mean diameter of between about 100 nm and about 200 nm.

In a particularly advantageous embodiment, the emulsion contains an encapsulating agent, which facilitates encapsulating the oil composition upon further processing of the emulsion (e.g., by spray drying). The encapsulating agent may be any edible substance capable of encapsulating the oil composition. Preferably, the encapsulation agent is predominantly a colloidal material. Such materials include starches, proteins from animal sources (including gelatins), proteins from plant sources, casein, pectin, alginate, agar, maltodextrins, lignin sulfonates, cellulose derivatives, sugars, saccharides, sorbitols, gums, and any combination thereof.

Suitable starches include: plant starches (e.g., CAPSUL® or HI-CAP® from National Starch & Chemical Corp., New York, N.Y.), other modified food starches, and any combination thereof. Preferably, the starch is CAPSUL® modified plant starch. Suitable proteins from animal sources include: gelatins (e.g., bovine gelatins, porcine gelatins (Type A or B) with different Bloom numbers, fish gelatins), skim milk protein, caseinate, and any combination thereof. Preferably, the animal protein is a gelatin. Suitable proteins from plant sources include: potato protein (e.g., ALBUREX® from Roquette Preres Societe Anonyme, Lestrem, France), pea protein, soy protein, and any combination thereof. Preferably, the plant protein is ALBUREX® potato protein. Suitable maltodextrins with a different dextrose equivalent include: maltodextrin 5, maltodextrin 10, maltodextrin 15, maltodextrin 20, maltodextrin 25, and any combination thereof. Preferably, the maltodextrin is maltodextrin 15. Suitable cellulose derivatives include: ethyl cellulose, methylethyl cellulose, hydroxypropyl cellulose, hydroxypropylmethyl cellulose, carboxymethylcellulose, and any combination thereof. Suitable saccharides include lactose, sucrose, or any combination thereof. Preferably, the saccharide is sucrose. Suitable gums include: acacia, locust bean, carragean, and any combination thereof. Preferably, the gum is gum acacia.

When the emulsion contains an encapsulating agent, the encapsulating agent may be dispersed in water by any convenient technology to form an aqueous phase. The aqueous phase may be a solution or a mixture depending on the properties of the components selected. The selected components may be dispersed by any convenient technology including: homogenizing, mixing, emulsifying, recirculating, static mixing, ultrasonication, stirring, heating, or any combination thereof. The viscosity of the resulting aqueous phase may then be adjusted, as desired, by the addition of water. The aqueous composition of the emulsion may optionally contain any other suitable material including but not limited to, those discussed above in reference to the nonaqueous composition. Preferably, the aqueous composition may include, an encapsulating agent, a film-forming agent, a plasticizer, a preservative, an antioxidant, or any combination thereof. Suitable preservatives include methyl paraben, propyl paraben, sorbic acid, potassium sorbate, sodium benzoate, and any combination thereof. Suitable antioxidants include sodium ascorbate, ascorbic acid, citric acid, and any combination thereof.

Preferably, the aqueous phase contains a modified food starch, such as octenyl succinyl starch (CAPSUL®), maltodextrin, and sodium ascorbate. Another preferred aqueous phase contains potato protein (ALBUREX®), maltodextrin 20, and sodium ascorbate. The selected components may be dissolved in water by any convenient technology, preferably stirring. The mixture is preferably homogenized until it is uniform and lump free. Preferably, the homogenization is carried out at a temperature between about 50° C. and about 75° C. The final viscosity of the resulting aqueous phase may then be adjusted to the desired viscosity, preferably about 250 cp to about 450 cp, more preferably about 300 cp to about 400 cp, even more preferably about 385 cp.

The emulsion may be formed by emulsifying the nonaqueous composition and the aqueous phase by any means, including homogenization, rotor-stator shear, high pressure shear and cavitation, high speed “cowles” or shear agitation, and any combination thereof. The volume and viscosity of the emulsion may preferably be adjusted by the addition of water after emulsification. Preferably, the nonaqueous and aqueous compositions are emulsified by homogenization. Preferably, the emulsion should not contain any mineral, transition metal, or peroxide.

As noted above, the emulsion may be incorporated or employed in producing other useful compositions, especially encapsulated oils, e.g., spray-dried powders. Generally, the encapsulated oil comprises an oil composition and an encapsulation agent encapsulating the oil composition, wherein the oil composition contains vitamin D3, 25-OH D3, or both dissolved in the oil in an amount between about 3% and about 50% by weight based on the total weight of the oil composition. The encapsulated oil may be produced by any convenient technology: e.g., drying an emulsion described above by any conventional technology, including spray drying, freeze drying, fluid bed drying, tray drying, adsorption, and any combination thereof. Preferably, the encapsulated oil is produced by spray drying an emulsion having an aqueous phase above containing an encapsulation agent; spray drying parameters are dictated by the physical characteristics desired in the final encapsulated oil. Such physical parameters include particle size, powder shape and flow, and water content. Preferably, the oil is in an amount less than about 30%, less than about 20%, less than about 10%, or less than about 3% by weight based on the total weight of the encapsulated oil. The encapsulated oil should have good flowability and the vitamin D3 and/or 25-OH D3 should be distributed homogeneously throughout the composition. Conveniently, the encapsulated oil is a powder. Any other suitable additive may be added to the encapsulated oil. One such additive may be a flow agent such as silicon dioxide, to increase the flowability of the encapsulated oil.

### Dosages

Daily. A composition according to this invention where the two active ingredients are to be administered separately, contains Vitamin D or 25-OH D3 in an amount from about 1 μg to about 50 μg, preferably about 5 μg and 25 μg. Alternatively, a single daily dosage having both Vitamin D and 25-OH D3 contains each active ingredient in an amount from about 1 μg to about 50 μg, preferably about 5 μg and 25 μg.

The dosage ratio of Vitamin D to 25-OH D3 may be from about 50:1 to about 1:50, more preferably from about 25:1 to about 1:25, and even more preferably from about 6:1 to about 1:6.

Multiple, separate dosages may be packaged in a single kit (or container). For example, the kit may be comprised of thirty separate daily dosages of both actives separately (i.e. 60 separate dosages), or combined (i.e. 30 dosages containing both active ingredients). Instructions for administering the dosages to a human may be included in the kit.

Weekly. A single weekly dosage contains Vitamin D or 25-OH D3 in an amount from about 7 μg to about 350 μg, and preferably from about 35 to 175 μg. Alternatively, a single weekly dosage may contain both Vitamin D and 25-OH D3 each in an amount from about 7 μg to about 350 μg, and preferably from about 35 to 175 μg. The dosage ratio of Vitamin D to 25-OH D3 may be from about 50:1 to about 1:50, more preferably from about 25:1 to about 1:25, and even more preferably from about 6:1 to about 1:6.

Monthly. A single monthly dosage contains Vitamin D or 25-OH D3 in an amount from 30 μg to about 1500 μg, preferably about 75 μg to about 500 μg. Alternatively, a single monthly dosage may contain both Vitamin D and 25-OH D3 each in an amount from 30 μg to about 1500 μg, preferably about 75 μg to about 500 μg. A kit may be comprised of one, two, three, four, five, six, seven, eight, nine, ten, eleven, or twelve weekly or monthly dosages.

Dosage ratios of Vitamin D to 25-OH D3 should range between 50:1 to about 1:50, more preferably from about 25:1 to about 1:25, and even more preferably from about 6:1 to about 1:6.

A study of the pharmacokinetics in humans of orally-administered spray-dried formulation of 25-OH D3, spray-dried formulation of vitamin D3, or both was initiated to investigate their physiological interactions. In particular, the shapes of their dose-response curves (which indicates the concentrations of vitamin D3 and 25-OH D3 in the circulation over a set time course, not simply the average or maximum concentration achieved) and the steady-state kinetics were of interest. In respect of the former point, it is important to investigate the change in shape of the dose-response curves when exposure is to both vitamin D3 and 25-OH D3. In respect of the latter point, it is also necessary to investigate their steady-state kinetics when dosing is less frequent than daily because this is the preferred regimen for groups that may have low compliance with taking daily supplements (such as the elderly).

The following non-limiting Examples are presented to better illustrate the invention.

### EXAMPLES

### Example 1

### Clinical Trial

### Formulation

Spray-dried formulation of 25-OH D3 was provided as a powder. In summary, 25-OH D3 and DL-α-tocopherol were dissolved in an oil of medium chain triglycerides, then emulsified into an aqueous solution of modified starch, sucrose, and sodium ascorbate. The emulsion was atomized in a spray dryer in the presence of silicon dioxide. The resulting powder was collected when water content (LDO) was less than 4% and sieved through 400 μm. It was packed and sealed in alu-bags, then stored in a dry area below 15° C. and used within 12 months of its manufacture.

Three separate lots were manufactured. In detail, a matrix was produced by mixing for 120 min in a FRYMIX processing unit with an anchor stirrer at 70° C. under vacuum and consisting of:

17.300 kg water (WBI)

13.460 kg modified food starch (CAPSUL HS)

3.270 kg sucrose

0.730 kg sodium ascorbate

An oil phase was prepared by mixing for 35 min in a double-walled vessel with propeller stirrer at 82° C. and consisting of:

0.550 kg BERGABEST MCT oil 60/40

0.049 kg calcifediol (HY-D USP)

0.183 kg DL-α-tocopherol

The oil phase was transferred to the matrix in the FRYMIX processing unit and was pre-emulsified with its internal colloid mill (60 min, 70° C.). The pre-emulsion was circulated through a high-pressure homogenizer (20 min). The emulsion with a viscosity of 60 mPa·s to 90 mPa·s at 70° C. was transferred over the high pressure pump to the spray nozzle. As fluidizing agent, silicon dioxide (SIPERNAT 320 DS) was fed into the tower. The spraying and drying parameters are listed below.

| Parameter | Spraying | Drying |
|---|---|---|
| Inlet air position | top of tower | top of tower |
| Inlet air feed | 1500 m³/h | 1400 m³/h |
| Inlet air temperature | 170° C. | heater switch off |
| IFB inlet air feed | 500 m³/h | 500 m³/h |
| IFB inlet air temperature | 65° C. | 50° C. |
| exhaust air position | bottom of the tower | bottom of the tower |
| fine powder recycling | to IFB | to IFB |
| emulsion feed rate | 50 kg/h | emulsion feed stopped |
| SiO₂ feed position | top of tower | SiO₂ feed stopped |
| SiO₂ acid feed rate | 100 g/h | SiO₂ feed stopped |

For each of the three lots of 25-OH D3, an average of 8.4 kg of spray-dried powder with about 0.25% content of 25-OH D3 was obtained. The other components of the formulation are: 73.2% modified food starch, 17.6% sucrose, 4.0% sodium ascorbate, 3.0% medium chain triglycerides, 1.0% silicon dioxide, and 1.0% DL-α-tocopherol.

Spray-dried formulation of vitamin D3 was provided as a powder. In summary, vitamin D3 and DL-α-tocopherol were dissolved in an oil of medium chain triglycerides, then emulsified into an aqueous solution of modified starch, sucrose, and sodium ascorbate. The emulsion was atomized in a spray dryer in the presence of silicon dioxide. The resulting powder was collected when water content (LOD) was less than 4% and sieved to remove big lumps. It was stored in a dry area below 15° C. and used within 12 months of its manufacture.

### Clinical Trial

### Subjects

Healthy, postmenopausal women (50 to 70 years of age) were recruited using informed consent and screened using the following criteria: serum 25-hydroxy vitamin D3 between 20 nmol/L and 50 nmol/L, body mass index between 18 kg/m² and 27 kg/m², blood pressure less than 146/95 mm Hg, serum calcium less than 2.6 nmol/L, fasting glucose less than 100 mg/dl, no high-intensity exercise more than three times per week, no treatment for hypertension, no use of high-dose vitamin D or calcium supplement or drug affecting bone metabolism (e.g., biphosphonate, calcitonin, estrogen receptor modulator, hormone replacement therapy, parathyroid hormone), and not visiting a “sunny” location during the study.

Subjects were randomly assigned to one of seven treatment groups (i.e., daily, weekly, bolus as single dose, and bolus as combination dose). Each group included five subjects. They are followed for four months in Zürich, Switzerland during the winter.

### Clinical Study

Pharmacokinetic characteristics of vitamin D3 and 25-OH D3 were studied. Equimolar quantities of both substances were investigated. The regimen was based on 20 μg/day (or its equivalent on a weekly basis) of 25-OH D3. For comparative purposes, it was necessary to administer equimolar quantities of either vitamin D3 or 25-OH D3. In respect to administration of vitamin D3, the dose was considered to be sufficient to overcome background variability and provide and efficacious dose to the participants.

Daily: 120 administrations

| | | |
|---|---|---|
| 1. | 25-OH D3 | 20 μg |
| 2. | Vitamin D3 | 20 μg (800 IU) |

Weekly: 16 administrations

| | | |
|---|---|---|
| 3. | 25-OH D3 | 140 μg |
| 4. | Vitamin D3 | 140 μg (5600 IU) |

Bolus: single administration

| | | |
|---|---|---|
| 5. | 25-OH D3 | 140 μg |
| 6. | Vitamin D3 | 140 μg (5600 IU) |

Bolus: combo administration

| | | |
|---|---|---|
| 7. | D3 and 25(OH)D3 | 140 μg (5600 IU) + 140 μg |

Hard gel capsules, which were packaged in bottles, contained either 20 μg or 140 μg of either spray-dried vitamin D3 or 25-OH D3 per capsule. Each dosage was consumed orally at breakfast. The duration of the study was four months for the “Daily” and “Weekly” groups. Subjects enrolled in the “Bolus” group consumed orally a single dosage at the second study visit.

Plasma concentrations of 25-OH D3 (e.g., peak and steady state) were determined by obtaining samples from the subjects at various times after the dosage was ingested. For screening purposes and to establish baseline values, a blood sample was obtained prior to enrollment into the study and the clinical laboratory measures vitamin D3, 25-OH D3, calcium, creatinine, albumin, and fasting glucose in the serum. On Monday of Week 1 of the study, pharmacokinetics of serum vitamin D3, 25-OH D3, and 1,25-dihydroxyvitamin D3; serum markers (i.e., vitamin D3, 25-OH D3, calcium, creatinine, albumin, PTH, GOT, GPT, ALP, triglycerides, HDL, LDL, total cholesterol, bALP, and fasting glucose); and urine markers (i.e., calcium, creatinine, and DPD) were assessed over 24 hours. Daily samples for the remaining days of Week 1 and Monday of Week 2 were taken to assess serum vitamin D3 and 25-OH D3, serum markers (i.e., calcium, creatinine, albumin), and urine markers (i.e., calcium, creatinine). The assessments continued on Monday of Weeks 3, 5, 7, 9, 11, 13 and 15. On Monday of Week 16, samples were taken to assess pharmacokinetics of serum vitamin D3, 25-OH D3, and 1,25-dihydroxyvitamin D3; serum markers (i.e., vitamin D3, 25-OH D3, calcium, creatinine, albumin, PTH, GOT, GPT, ALP, triglycerides, HDL, LDL, total cholesterol, bALP, and fasting glucose); and urine markers (i.e., calcium, creatinine, and DPD).

### Results

Table 1 shows bone resorption markers deoxypyridinoline (DPD) and bone-specific alkaline phosphatase (BAP) after daily and weekly treatment with 25-OH D3 (20 μg per day; 140 μg per week, respectively) and daily and weekly treatment with Vitamin D3 (20 μg per day; 140 μg per week, respectively). Treatment duration was 4 months. Values are GLM (general linear model) least square means after 4 months adjusted for baseline bone resorption markers, age and body mass index for 25-OH D3 and Vitamin D3.

TABLE 1

| | Bone resorption markers | |
|---|---|---|
| | Daily/weekly Vitamin | Daily/weekly |
| | D3 | 25-OH D3 |
| DPD (in nmol/mmol creatine) | 9.8 | 8.2 |
| BAP (in IU/L) | 76.5 | 74.7 |

Table 2 shows the relative change in bone resorption markers deoxypyridinoline (DPD) and bone-specific alkaline phosphatase (BAP) after daily and weekly treatment with 25-OH D3 (20 μg per day; 140 μg per week, respectively) compared to daily and weekly treatment with Vitamin D3 (20 μg per day; 140 μg per week, respectively). Treatment duration was 4 months. Values are GLM (general linear model) least square means given as % improvement adjusted for baseline bone markers, age and body mass index for 25-OH D3 versus Vitamin D3 are given as % reduction of bone resorption markers after repeated measurement analysis over 13 visits adjusted for baseline bone resorption markers and time for 25-OH D3 versus Vitamin D3.

| | Bone resorption markers (Change |
|---|---|
| | vs. Daily/weekly Vitamin D3 in %) |
| | Daily/weekly 25-OH D3 |
| DPD | −17.0 |
| BAP | −2.4 |

These data demonstrate that daily or weekly treatment with 25-OH D3 surprisingly results is much stronger reductions of bone resorption markers compared to consumption of identical dosages of Vitamin D3. After treatment with 25-OH D3 subjects displayed more pronounced reductions in bone resorption markers compared to before treatment and compared to treatment with Vitamin D3. The reduction of deoxypyridinoline (DPD) and bone-specific alkaline phosphatase (BAP) in subjects treated with 25-OH D3 versus Vitamin D3 was 17.0 and 2.4%, respectively, an effect size that is clinically relevant and represents a significant benefit for subjects in all age groups.

A reduction of markers for bone resorption such as deoxypyridinoline (DPD) and bone-specific alkaline phosphatase (BAP) indicates that the balance between bone resorption and bone formation is shifted to formation which ultimately results in accretion of bone mass or at least decreased loss of bone mass. In conclusion, treatment with 25-OH D3 surprisingly resulted in a very effective reduction of bone resorption markers compared to Vitamin D3 indicating that net bone formation is improved following treatment with 25-OH D3.

TABLE 3 shows the increase in plasma 25-OH D levels after a dosage of 140 μg 25-OH D3, a dosage of 140 μg Vitamin D or the combined dosage of 140 μg 25-OH D3+140 μg Vitamin D. Blood samples were obtained according to the depicted time schedule.

TABLE 3

| | Plasma 25-OH D (Change vs. baseline in nmol/L) | | |
|---|---|---|---|
| Time | | | 140 μg 25-OH D3 + |
| (hours) | 140 μg 25-OH D3 | 140 μg Vitamin D3 | 140 μg Vitamin D3 |
| 0 | 0.0 | 0.0 | 0.0 |
| 2 | 28.5 | 1.5 | 41.2 |
| 4 | 47.7 | 2.2 | 61.6 |
| 6 | 58.2 | 3.2 | 64.6 |
| 8 | 60.2 | 5.2 | 62.4 |
| 10 | 57.7 | 6.2 | 63.1 |
| 12 | 58.9 | 6.7 | 63.9 |
| 25 | 42.2 | 8.0 | 46.2 |
| 49 | 34.4 | 11.7 | 43.2 |
| 73 | 29.7 | 13.0 | 39.7 |
| 97 | 22.2 | 12.7 | 33.9 |
| 206 | 17.5 | 14.0 | 31.9 |
| 374 | 12.7 | 13.0 | 20.7 |
| 708 | 7.5 | 13.7 | 14.2 |

As shown above, there was a synergistic increase in the plasma 25-OH D response after a combined administration of 140 μg 25-OH D3+140 μg Vitamin D3. The effect was especially pronounced during the first 6 hours. Furthermore, a combined administration produced sustained increase in plasma 25-OH D levels by at least 30 nmol/L from 2-206 hours (i.e until 8.5 days, or over 1 week). After administration of 140 μg 25-OH D3, an increase in plasma 25-OH D levels by at least 30 nmol/L was observed between 4-49 hours only, whereas no increase of that magnitude was observed after administration of 140 μg Vitamin D3 alone.

Therefore, a combined administration of 140 μg 25-OH D3+140 μg Vitamin D3 provides two significant advantages: It results in a rapid and synergistic plasma response of 25-OH D and it leads to an unexpectedly pronounced and long plateau of plasma 25-OH D levels. These are especially important goals of treatment of Vitamin D deficiency; fast correction of suboptimal Vitamin D status and a long and stable plasma concentration to ensure sufficient supply of all Vitamin D dependent tissues.

Treatment with 25-OH D3 reduces bone resorption makers more effectively compared to an equal dosage of Vitamin D3. This results in a more pronounced shift of the balance between bone resorption and bone formation towards bone formation and, therefore, to improved bone health. Due to the synergistic increase in plasma 25-OH D levels after combined administration of 25-OH D3 and Vitamin D3 bone formation is accelerated even further which results in stronger benefits for bone health.

1. A method of maintaining bone health, and/or preventing or treating osteoporosis, rickets and osteopenia, comprising administering vitamin D and 25-OH D3 to a human.

2. The method according to claim 1, wherein vitamin D and 25-OH D3 are administered separately.

3. The method according to claim 1, wherein vitamin D and 25-OH D3 are administered together.

4. The method according to claim 1, wherein vitamin D and 25-OH D3 are administered once daily.

5. The method according to claim 1, wherein vitamin D and 25-OH D3 are administered once weekly.

6. The method according to claim 1, wherein vitamin D and 25-OH D3 are administered once monthly.

7. The method according to claim 1 further comprising administering one or more additional osteoporosis medicaments to the human.

8. A bone health promoting food, functional food, food supplement or nutraceutical suitable for human consumption containing 25-OH D3, and preferably a combination of Vitamin D and 25-OH D3.

9. Use of 25-OH D3 and Vitamin D in the manufacture of a medicament, food, functional food, food supplement or nutraceutical which promotes bone health in a human.
